(19) United States
(12) Patent Application Publication
(10) Pub. No.: US 2004/0199092 A1
(43) Pub. Date: Oct. 7, 2004

(54) Adjustable protective covers for medical devices and wounds

(76) Inventors: Andre Biewend, Longwood, FL (US); Ronald E. Caponi, Longwood, FL (US)

Correspondence Address: Lara A. Northrop, Pietragallo, Bosick & Gordon, One Oxford Centre, 38th Floor, 301 Grant Street, Pittsburgh, PA, 15219, US

(21) Appl. No.: 10/745,201

(22) Filed: Dec. 23, 2003

Related U.S. Application Data
(60) Provisional application No. 60/436,025, filed on Dec. 23, 2002.

(51) Int. Cl. A61F 5/00

(52) U.S. Cl. 602/3

(57) ABSTRACT

An adjustable protective cover for covering a patient's appendage and a medical device and/or a wound is disclosed. The cover includes an elongated water resistant flexible bag and at least two fastening elements disposed on the flexible bag that engage to shorten the net length and/or width of the flexible bag. A sealing band is also disclosed that engages the opening of the flexible bag and provides a water resistant seal without creating a tourniquet on the patient's appendage.

### CROSS-REFERENCE TO RELATED APPLICATION

This application claims the benefit of U.S. Provisional Patent Application Serial No. 60/436025 filed Dec. 23, 2002, which is incorporated herein by reference.

### FIELD OF THE INVENTION

The present invention relates to protective covers for a patient's appendage having a medical device or wound, and more particularly relates to adjustable protective covers for arm and leg casts, wounds, PICC Lines, Active IV sites, Infusion Therapy Sites, diabetic ulcers and/or compression wraps.

### BACKGROUND INFORMATION

Medical devices such as plaster casts, splints, braces, surgical dressings, prostheses, compression wraps, long-term active IV sites and PICC lines often must be kept dry for the entire duration the device is worn by a patient. This can make common activities like showering, washing and walking in rain or snow difficult for patients using these types of long-term devices. Broken bones and severally injured appendages are typically restrained by a plaster cast for a period of time from four weeks to ten weeks. When plaster that is typically used to form a cast around the broken or injured appendage gets wet, the plaster becomes soft and deteriorates thereby weakening the structural integrity of the cast and increasing the probability of mold or mildew growth. Furthermore, when water resistant materials are employed, the patient often experiences discomfort when the afflicted area becomes wet. Additionally, an unpleasant odor will develop due to fungal growth beneath the casting material. This situation is further compounded, and becomes increasing dangerous for the patient, when an open wound or laceration, exists beneath the casting material thereby greatly increasing the likelihood of infection. Active IV sites, peripherally inserted centralline catheters (“PICC lines”) and Mid-lines (collectively, “Infusion Therapy Sites”) require an insertion into a central vein to remain open for a period of time from days to years. When these sites become wet, they tend to promote bacterial growth beneath the dressing. Typically, an Infusion Therapy Site that becomes wet requires the medical device to be removed from the patient within five to seven days and replaced by another assembly. The replacement process can be expensive, time consuming and requires additional medical professional application.

A common practice to assist in protecting long-term medical devices from moisture is to cover the outside surface of the afflicted area with a sheet of plastic to prevent water from reaching the surface of the device. This arrangement requires a seal of some kind around the upper and lower portions of the device to prevent water from leaking between the patient's appendage and the long-term medical device. Traditional devices, such as buckles and inches, which generate a seal sufficient to prevent water leakage often result in a tourniquet, which cuts off or significantly curtails blood flow to the injured appendage. In order for active Infusion Therapy Sites to function properly, blood flow to and from the injured area cannot be disrupted. Blood flow is also a major issue concerning patients with wounds, especially diabetic ulcers and compression bandages. Accordingly, tourniquet restrictions are infeasible means for waterproofing the injured area. If the traditional devices are sufficiently loosened such that blood flow to the injured area is not restricted, water leakage occurs.

Other prior designs for waterproofing a long-term medical device and the surrounding area of a patient include using a water resistant bag having an elastic edge wrapped with an elastic sealing band. Although this design is suitable in some applications, there are many other circumstances in which this design is not optimal. For example, the prior design may not be long enough to reach high line PICC insertions, and may not be long enough or wide enough to cover surgical procedures performed on the elbow. By simply making a longer bag, the bag would be too long for smaller patients, and some patients do not require a longer bag since their affected area occurs lower on the arm or leg. In addition, merely making the bag longer or wider generates excess material that can cause discomfort to the patient and making the unit more cumbersome to use. In addition, excess material can increase the chances that a patient will “catch” and rip the bag on an object thus creating the potential for compromising the water resistant integrity of the product.

Typically, waterproofing bags are sold to hospitals and medical professionals in generally one size for all adolescents and adults. Requiring hospitals and medical professionals to stock water resistant bags in multiple sizes is undesirable due to stocking, ordering and sizing concerns. Accordingly, a need remains for a water resistant device capable of protecting a long-term medical device from moisture that is sufficiently large to cover injuries that occur high on an appendage, such as high line PICC insertions and surgical procedures performed on the elbow, which does not add an excess of additional material to the device.

The present invention has been developed in view of the foregoing.

### SUMMARY OF THE INVENTION

An adjustable protective cover for covering a patient's appendage and a medical device and/or a wound is disclosed. The cover includes an elongated water resistant flexible bag and at least two fastening elements disposed on the flexible bag that engage to shorten at least one dimension of the flexible bag. A sealing band is also disclosed that engages the opening of the flexible bag and provides a water resistant seal without creating a tourniquet on the patient's appendage.

An aspect of the present invention is to provide an adjustable protective cover for a medical device or wound comprising an elongated water resistant flexible bag adjustable to an extended position and a folded position and having an opening on at least one end capable of housing at least part of a patient's appendage and a medical device or wound in the interior of the flexible bag.

Another aspect of the present invention is to provide an adjustable cover system for a medical device or wound comprising an elongated water resistant flexible bag adjustable to an extended position and a folded position and having an opening on at least one end capable of housing at least part of a patient's appendage and a medical device or wound in the interior of the flexible bag; at least one first bag fastening element disposed on a surface of the flexible bag; at least one second bag fastening element disposed on a surface of the flexible bag, the second bag fastening element capable of being engaged by the first bag fastening element; at least one sealing band anchor disposed on an exterior surface of the flexible bag; and an elongated water-resistant flexible substantially flat sealing band having sufficient length to be wrapped around the patient's appendage at least once, the sealing band comprising at least one substantially flat side having at least one portion capable of engaging the sealing band anchor, and a sealing band connector disposed on the substantially flat side capable of attaching to an area on an opposing side of the sealing band or the flexible bag to substantially seal the open end of the flexible bag against the intrusion of moisture.

Yet another aspect of the present invention it to provide a method of using an adjustable cover for a medical device or wound comprising providing an elongated water resistant flexible bag adjustable to an extended position and a folded position and having an opening on at least one end; inserting a patient's appendage through the opening into the flexible bag; sealing the opening against the patient's appendage; and folding a portion of the bag from the extended position to the folded position.

### BRIEF DESCRIPTION OF THE DRAWINGS

FIG. 1 illustrates an adjustable protective cover for an arm injury in accordance with an embodiment of the present invention.

FIG. 2A illustrates a top view of a sealing band in accordance with an embodiment of the present invention.

FIG. 2B illustrates a bottom view of a sealing band in accordance with an embodiment of the present invention.

FIG. 3A illustrates an adjustable protective cover for an arm injury having a defined thumb area in accordance with an embodiment of the present invention.

FIG. 3B illustrates an adjustable protective cover for an arm injury having two attachment systems and a defined thumb area in accordance with an embodiment of the present invention.

FIG. 4A illustrates an adjustable protective cover for an arm injury having an elastic edge on the open end in accordance with an embodiment of the present invention.

FIG. 4B illustrates an adjustable protective cover for an arm injury having an elastic edge on the open end and a defined thumb area in accordance with an embodiment of the present invention.

FIG. 5 illustrates an adjustable protective cover as used on an arm injury, showing the process of forming a seal with a sealing band in accordance with an embodiment of the present invention.

FIG. 6 illustrates an adjustable protective cover as used on an arm injury, showing the completed seal formed by the sealing band in accordance with an embodiment of the present invention.

FIG. 7 illustrates an adjustable protective cover as used on an arm injury, showing the process of adjusting the flexible bag by changing the extended length of the flexible bag into the folded length of the flexible bag in accordance with an embodiment of the present invention.

FIG. 8A illustrates an adjustable protective cover for a leg injury in accordance with an embodiment of the present invention.

FIG. 8B illustrates an adjustable protective cover for a leg injury in accordance with an embodiment of the present invention.

FIG. 9A illustrates a top view of a sealing band in accordance with an embodiment of the present invention.

FIG. 9B illustrates a bottom view of a sealing band in accordance with an embodiment of the present invention.

FIG. 10A illustrates an adjustable protective cover as used on a leg injury, showing the process of forming a seal with a sealing band in accordance with an embodiment of the present invention.

FIG. 10B illustrates an adjustable protective cover as used on a injury, showing the process of forming a seal with a sealing band in accordance with an embodiment of the present invention.

FIG. 11 illustrates an adjustable protective cover as used on a leg injury, showing the completed seal formed by the sealing band in accordance with an embodiment of the present invention.

FIG. 12A illustrates an adjustable protective cover as used on a leg injury, showing the process of adjusting the flexible bag by changing the extended length of the flexible bag into the folded length of the flexible bag in accordance with an embodiment of the present invention.

FIG. 12B illustrates an adjustable protective cover as used on a leg injury, showing the process of adjusting the flexible bag by changing the extended length of the flexible bag into the folded length of the flexible bag in accordance with an embodiment of the present invention.

### DETAILED DESCRIPTION

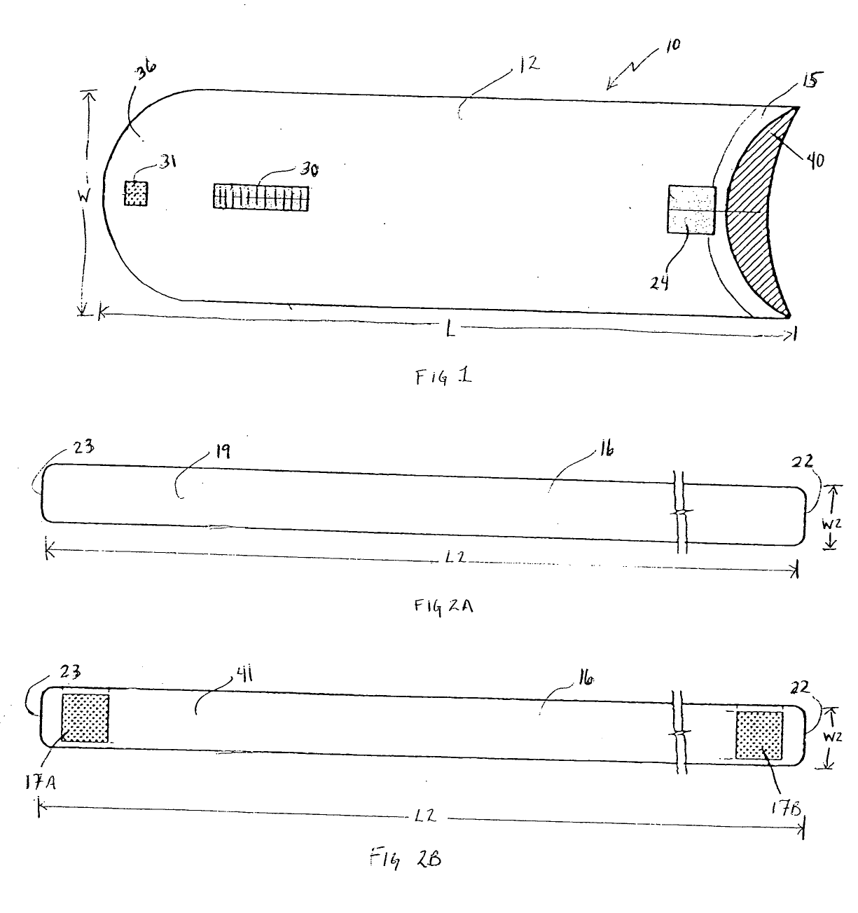

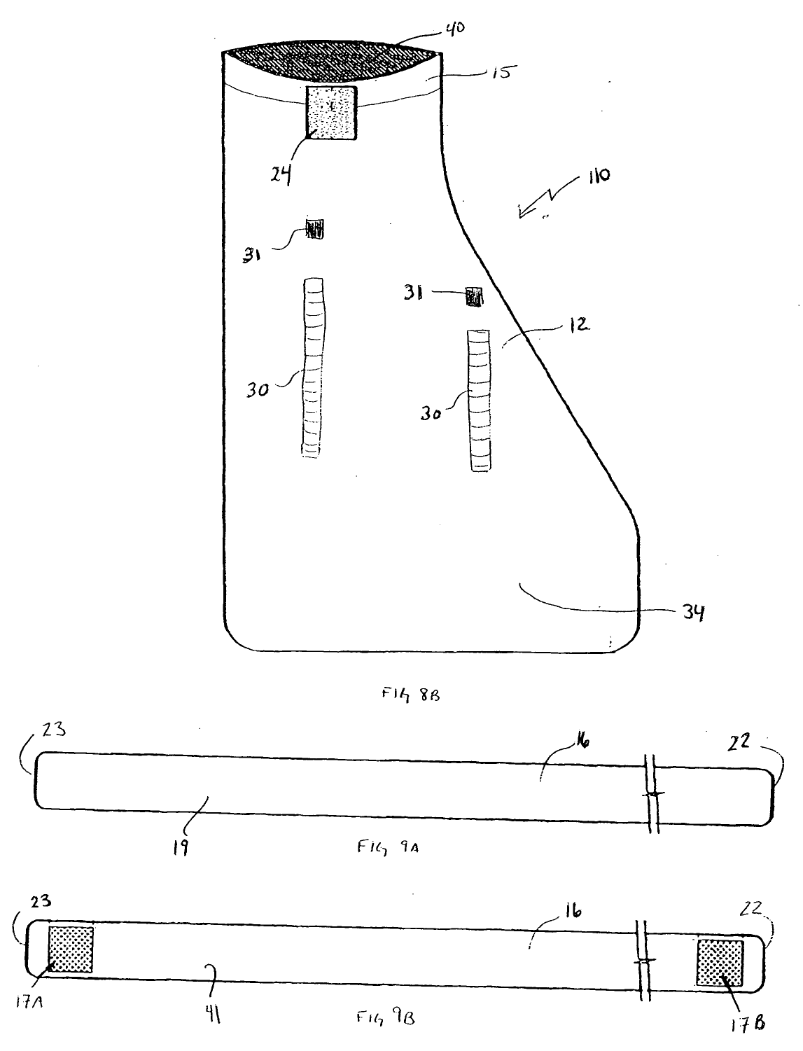

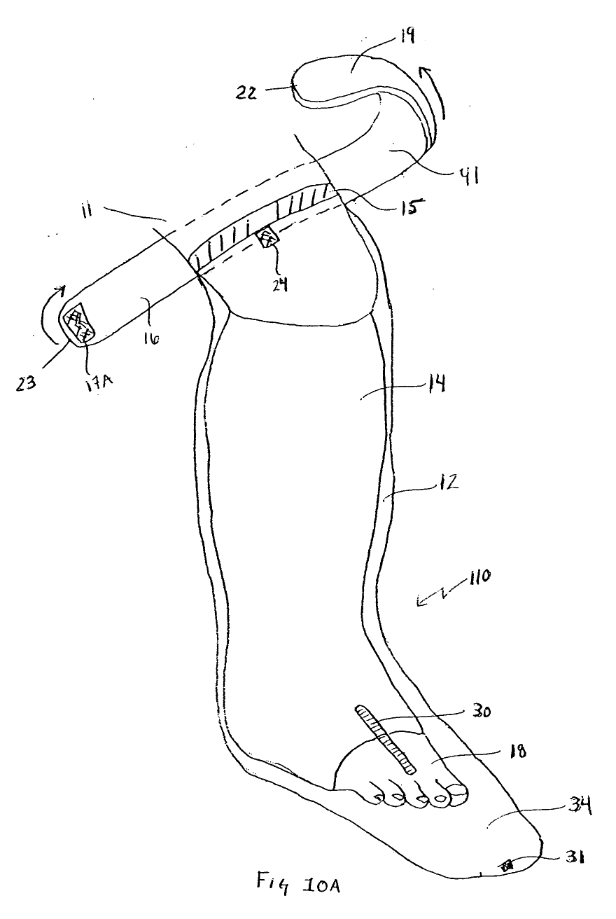

FIG. 1 schematically illustrates an adjustable protective cover 10 for an arm injury in accordance with an embodiment of the present invention. FIGS. 8A and 8B schematically illustrate an adjustable protective cover 110 for a leg injury in accordance with another embodiment of the present invention. In each embodiment, the adjustable protective cover 10, 110 includes an elongated water resistant flexible bag 12 that can be placed over a patient's appendage having an open wound and/or a long-term medical device in contact with or connected to the patient's appendage. As used herein, the term “water resistant” means that the material resists at least some water penetration. As used herein, the term “patient” means an adult of pediatric human, or an animal such as dogs, cats, horses and the like. The adjustable protective cover 10, 110 can be removed after use and reused a plurality of times. As used herein, the term “long-term medical device” means any medical implement used to help treat an injury that remains connected to, or in contact with, a part of a patient's body for an extended period of time. Examples of long-term medical devices include casts such as plaster casts and synthetic casts, braces, PICC lines, active IV sites, bandages, surgical dressings, compression bandages and wraps, prostheses and splints. As used herein, the term “wound” means any damage to a patient's body requiring at least some protection from external moisture or contaminant. Examples of wounds include lacerations, sutures, burns, rashes, allergic reactions, diabetic ulcers, skin grafts, and the like. As used herein, the term “extended period of time” means a duration of time throughout which keeping a long-term medical device or wound protected from external moisture or contaminants would be difficult, expensive or uncomfortable. An extended period of time could last for a day, several days, several weeks, several months or years. For example, a plaster cast may remain in contact with a patient's body for from about four weeks to about two months and a PICC line may remain connected to a patient's body for from about one week to a period of years.

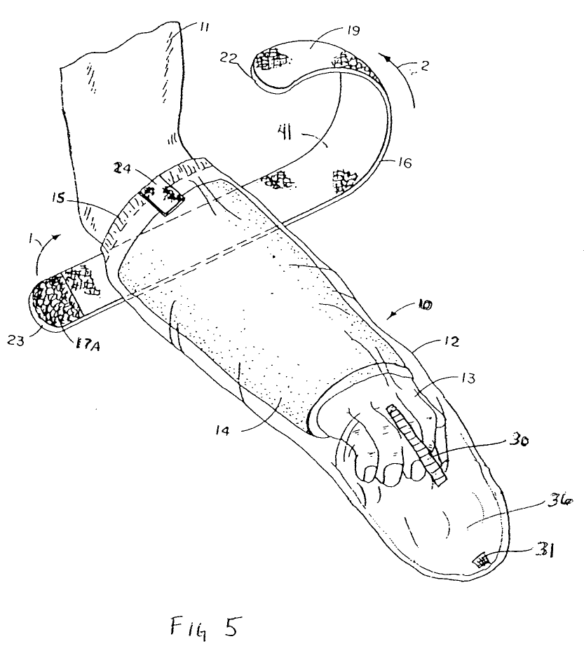

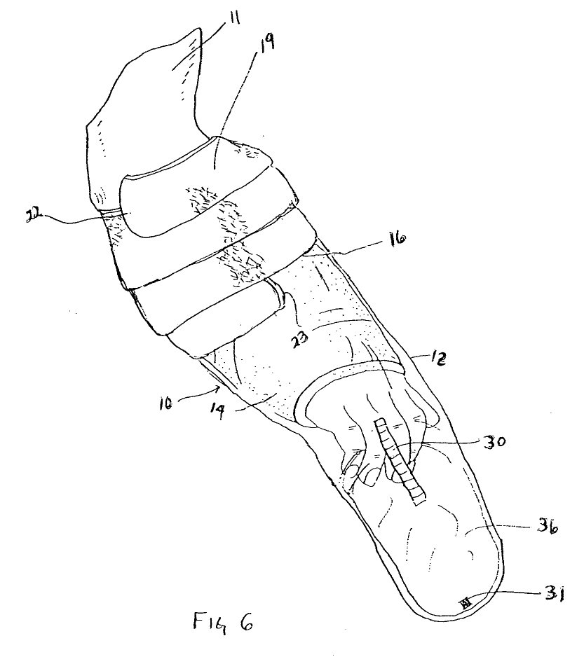

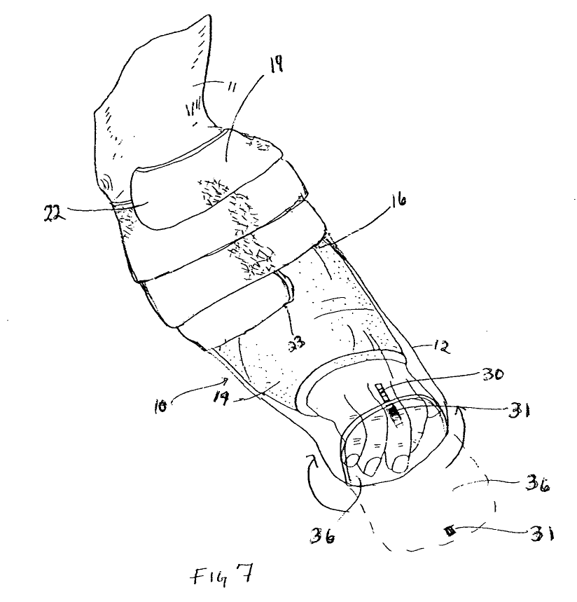

The flexible bag 12 can be slid over a patient's appendage, such as an arm or a leg, having an open wound or a long-term medical device. As shown in FIGS. 5-7 and 10A-12B, the flexible bag 12 has an opening 40 that can be slid over a patient's appendage that is surrounded by a medical device such as a cast 14. In another embodiment, it is possible to slide the flexible bag 12 over a patient's appendage and active IV or PICC line, covering both the appendage and the active IV or PICC line with the flexible bag 12 without dragging the opening over the afflicted area thus the opening is able to be lifted over the afflicted area.

The flexible bag 12 can be formed from any flexible water resistant material. The flexible bag 12 may comprise a polymer sheet such as PVC, latex, poly-vinyl taffeta, waterproof canvas and any other waterproofing materials. In one embodiment, a transparent, semi-transparent or translucent PVC sheet is used to form the flexible bag 12. The flexible bag 12 may be made of a reusable material. In another embodiment, a heat-shrinkable polymer can be used to form the flexible bag 12. In one embodiment, a polymer material is extruded into a bag shape to form the flexible bag 12. In another embodiment, flat polymer sheet can be cut to shape and heat-sealed along the seam to form the flexible bag 12.

The flexible bag 12 is sized to allow at least a portion of a patient's appendage, having a wound and/or a long-term medical device to be inserted into the interior of the bag and housed within the interior of the bag. In the case of an injury to a patient's arm, the hand 13 and at least a portion of the arm is inserted into the flexible bag 12. In the case of an injury to a patient's leg, the foot and at least a portion of the leg are inserted into the flexible bag 12. The flexible bag 12 has an extended position and a folded position.

In one embodiment, the extended length of the flexible bag 12, represented by the letter L in FIG. 1, is from about 5 inches to about 48 inches. In another embodiment, the extended length of the flexible bag 12 used for an adult arm is from about 24 inches to about 36 inches. The extended length of the flexible bag 12 used for a pediatric arm is from about 5 inches to about 18 inches. In another embodiment, the extended length of the flexible bag 12 used for an adult leg is from about 24 inches to about 48 inches. The extended length of the flexible bag 12 used for a pediatric leg is from about 7 inches to about 22 inches. The extended width of the flexible bag 12, represented by the letter W in FIG. 1, is from about 2 inches to about 18 inches. In another embodiment, the extended width of the flexible bag 12 used for an adult arm is from about 3 inches to about 9 inches. The extended width of the flexible bag 12 used for a pediatric arm is from about 2 inches to about 6 inches. In another embodiment, the extended width of the flexible bag 12 used for an adult leg is from about 4 inches to about 26 inches. The extended length of the flexible bag 12 used for a pediatric leg is from about 3 inches to about 9 inches. The thickness of the material used to form the flexible bag 12 can be from about 2 mils to about 7 mils. Preferably, the material used to form the flexible bag 12 has a thickness of from about 3 mils to about 4 mils.

The flexible bag 12 can be folded into a folded position that reduces the net length and/or width of the bag by any desired amount. In one embodiment, the net folded length of the flexible bag 12 is from about less than 2 inches to about 20 inches smaller than the extended length of the bag. In another embodiment, the net folded length of the flexible bag 12 is from about 6 inches to about 12 inches smaller than the extended length of the bag. In another embodiment, the net folded width of the flexible bag is from about less than 2 inches to about 12 inches smaller than the extended width of the bag.

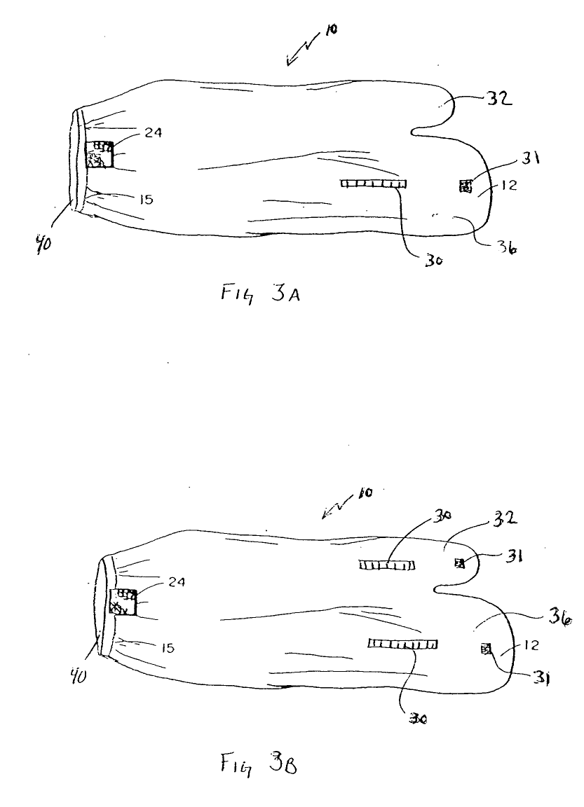

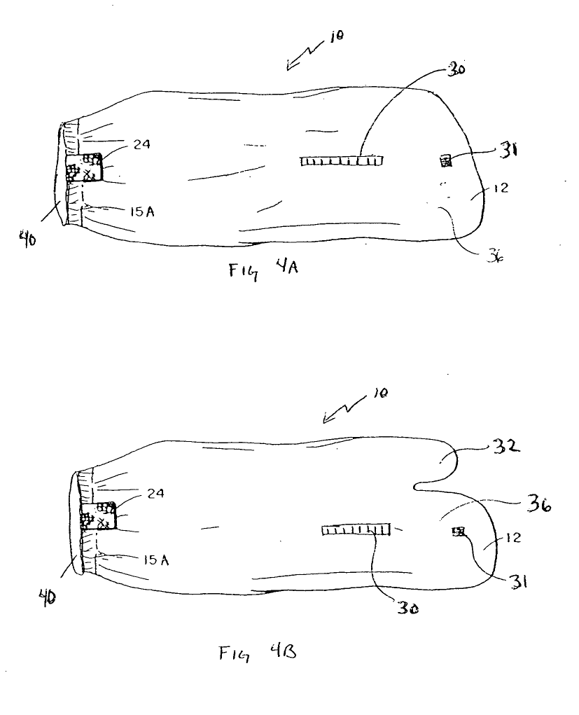

As shown in FIGS. 1 and 4A, the flexible bag 12 may comprise a standard bag configuration in which all of the patient's digits are housed in the finger area 36 of the flexible bag 12. As shown in FIGS. 3A, 3B and 4B, the flexible bag 12 may optionally comprise a finger portion 36 and a thumb portion 32. In this embodiment, the patient's fingers are housed in the finger area 36 of the flexible bag 12 and the patient's thumb is housed in the thumb portion 36 of the flexible bag 12. The separate finger portion 36 and thumb portion 32 allow a user to have increased dexterity for activities such as showering and bathing.

The flexible bag 12 has an annular edge 15 adjacent the opening 40. As shown in FIGS. 3A and 3B the annular edge 15 comprises a finished edge. In one embodiment, the annular edge 15 is slightly gathered to define a narrower opening 40 that the remainder of the flexible bag 12. In another embodiment, the annular edge 15 may be lined with a soft material to increase comfort and decrease skin irritation. The soft material may be attached to the flexible bag 12 through any attachment means. For example, the soft material may be sewn, glued, heat attached or otherwise adhered to the annular edge 15.

In another embodiment, as shown in FIGS. 4A, 4B and 5, the annular edge 15 can comprise an elastic material 15A. The elastic material 15A can help to hold the flexible bag 12 in place over the patient's appendage and can also assist in sealing the flexible bag 12, as is discussed herein. Elastic material 15A can be sewn, glued, heat attached or otherwise adhered onto the annular edge 15 of the flexible bag 12. In one embodiment, the elastic material 15A is pulled tight prior to being sewn to the annular edge 15 of the flexible bag 12 such that the opening of the bag naturally shrinks or contracts after the elastic material 15A is sewn to the annular edge 15. In another embodiment, the flexible bag 12 having elastic material 15A attached to the annular edge 15 has a bag opening that is from about 3 inches to about 5 inches when at rest in a contracted state and from about 6 inches to about 14 inches when pulled tight to an extended position.

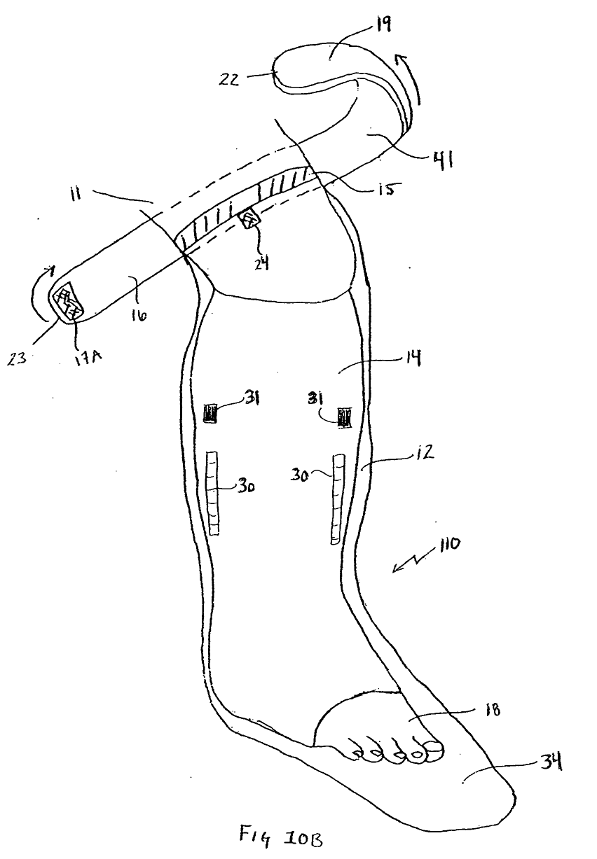

The flexible bag 12 further comprises a sealing band anchor 24 located adjacent the annular edge 15 of the bag opening 40. The sealing band anchor 24 fastens the sealing band 16 as shown in FIGS. 2A, 2B, 9A and 9B to the flexible bag 12. In one embodiment, the sealing band anchor 24 comprises one half of any two-piece sealing unit that can be attached and detached from the other half of the two-piece unit. Examples of sealing band anchors 24 can comprise looped material or hooked material, such as Velcro, a male end of a snap, a female end of a snap, a button, an eye, a hook, reusable adhesives and the like. In another embodiment, the sealing band anchor 24 semi-permanently attaches the sealing band 16 to the flexible bag 12. As used herein, the term “semi-permanently” means that the sealing band is connected to the sealing band anchor such that it resists at least some degree of oppressive force. In another embodiment, the sealing band anchor 24 can comprise grommets or other permanent fasteners. In one embodiment, sealing band 16 comprises a water-resistant material.

In another embodiment, the sealing band anchor 24 is a square of looped material having dimensions of from about one inch×one inch to about three inches×three inches. In another embodiment, the sealing band anchor 24 is located about one-half inch from the opening 40 of the flexible bag. In another embodiment, the sealing band anchor 24 overlaps the annular edge 15. In another embodiment, the sealing band anchor 24 is heat sealed, sewn, glued or otherwise attached to the flexible bag. In yet another embodiment, the sealing band anchor 24 is heat sealed to the flexible bag and sewn under the elastic material 15A on the annular edge 15.

As shown in FIGS. 2A and 2B and 9A and 9B, an elongated, substantially flat sealing band 16 is made of an elastic, resilient water resistant material. The sealing band 16 has at least one substantially flat side 41 that is capable of engaging a patient's skin to form a seal around the annular edge 15 of the flexible bag 12. In one embodiment, the substantially flat side 41 of the sealing band 16 comprises neoprene, waterproof foamed materials, such as those used to produce wet suits for scuba divers, latex straps, silicone based straps or seals and/or PVC bands. Neoprene is particularly preferred because it is durable, non-allergenic, latex-free and forms an improved water resistant seal without skin irritation. In another embodiment, the opposing side 19 of the sealing band can comprise a plush material such as cloth or synthetic materials having a loop structure.

In another embodiment, the sealing band 16 comprises at least one sealing band connector 17 that is capable of attaching the edges 22 and 23 of the sealing band 16 to another portion of the sealing band 16 or a portion of the flexible bag 12. The sealing band connector 17 may be disposed on the flat side 41 of the sealing band 16 and may engage a portion of the opposing side 19 of the sealing band 16 to form a seal around the patient's appendage.

In another embodiment, the sealing band 16 has an opposing side 19 comprising a looped material, and a substantially flat side 41 having hooked material 17A attached to a first end 23 of the sealing band 16 and hooked material 17B attached to a second end 22 of the sealing band 16. The opposing side 19 may comprise looped material throughout the entire length of sealing band 16. It is herein understood that the orientation of the sealing band 16 may be changed and that the specific orientation described herein is only for purposes of explanation and is not limiting of the present invention.

In one embodiment, the sealing band 16 has a length, as represented by “L2” in FIGS. 2A and 2B, of from about 3 inches to about 80 inches. In another embodiment, the length of the sealing band 16 used for an adult arm is from about 12 inches to about 60 inches. The length of the sealing band 16 used for a pediatric arm is from about 3 inches to about 18 inches. In another embodiment, the length of the sealing band 16 used for an adult leg is from about 18 inches to about 80 inches. The length of the sealing band 16 used for a pediatric leg is from about 3 inches to about 20 inches. In another embodiment, the sealing band 16 has a width, as represented by “W2” in FIGS. 2A and 2B, of from about 1 inch to about 4 inches. In another embodiment, the width of the sealing band 16 used for an adult arm is from about 1 inch to about 4 inches. The width of the sealing band 16 used for a pediatric arm is from about 1 inch to about 3 inches. In another embodiment, the width of the sealing band 16 used for an adult leg is from about 1 inch to about 4 inches. The width of the sealing band 16 used for a pediatric leg is from about 1 inch to about 3 inches. The thickness of the material used to form the sealing band 16 is from about 0.5 mm to about 2 cm. In another embodiment, the thickness of the material used to form the sealing band 16 is from about 3 mm to about 4 mm.

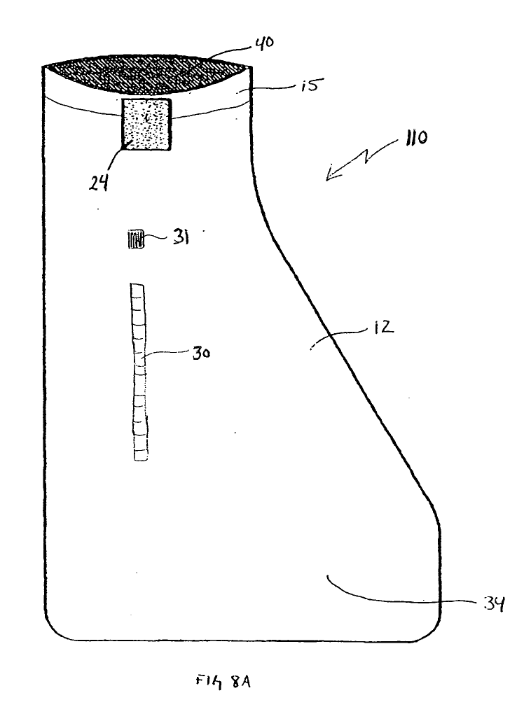

When the sealing band 16 is wrapped around a patient's appendage 11 over the annular edge 15 of the flexible bag 16, as shown in FIG. 5, the hook material 17A or 17B can be attached to the sealing band anchor 24 and pulled to stretch the sealing band 16 around the patient's appendage 11 in multiple overlapping coils. In order to effectuate a water resistant seal, a first end 23 of the sealing band 16 is first wrapped around the patient's appendage 11 in a first direction and fastened to the sealing band anchor as shown in FIG. 5. The second end 22 is subsequently wrapped around the patient's appendage 11 in a second direction that is generally opposite from the first direction. As the second end 22 is wrapped around the appendage 11, each successive wrap or coil overlaps at least a part of the previous coil of the sealing band 16, thereby creating a “shingling effect.” The wrapping of the sealing band 16 around the appendage to engage another portion of the sealing band or flexible bag does not have a tourniquet effect or substantially restrict blood flow to the injured area of the appendage. Accordingly, this sealing method can be used for patients having active wounds, diabetic ulcers, compression wraps/bandages, Infusion Therapy Sites, IV sites or PICC lines, whereas conventional sealing systems requiring tape, buckles or cinches cannot. In one embodiment, the substantially flat side 41 is adjacent the skin of the patient and the opposing side having a looped material 19 is located on the exterior of the coiled sealing band 16. As shown in FIG. 6, once the remaining length of the sealing band 16 is wound around the patient's appendage, the hooked material 17B is attached to the looped material 19 of the top portion of the sealing band 16.

After the sealing band 16 has been fastened to the flexible bag 12, an area of excess flexible bag material, such as finger portion 36 and/or thumb portion 32, often remains loose. Accordingly, flexible bag 12 also comprises a length and/or width adjusting system that is capable of altering the dimensions of the flexible bag 12 once a patient's appendage and long-term medical device or wound is positioned within the interior of the flexible bag 12. The length and/or width adjusting system comprises at least one first fastening element 30 and at least one second fastening element 31 capable of engaging the first fastening element 30 and forming a temporary or permanent seal.

In one embodiment of the present invention, the first fastening element 30 comprises a looped material, such as Velcro, and the second fastening element 31 comprises a hooked material, such as Velcro, that is capable of engaging the looped material first fastening element 30. In another embodiment, the first fastening element 30 and second fastening element 31 can comprise other sealing systems such as adhesive or tape means, snaps, buttons, hook and eye systems, zipper systems, magnetic systems and the like. In another embodiment, the first fastening element 30 comprises a strip of looped material having a length of from about 2.5 cm to about 30 cm, and a width of from about 5 mm to about 4 cm. The looped material is positioned on the flexible bag 12 at an area about 3 inches to about 12 inches from the bottom of the closed end of the flexible bag 12, such as in the finger portion 36. The second fastening element 31 comprises a section of hooked material having a length of from about 5 mm to about 4 cm and a width of from about 5 mm to about 4 cm. The second fastening element is located on the same side of the flexible bag 12 as the first fastening element 30. In one embodiment, the second fastening element 31 is located on the flexible bag 12 at an area about one-half inch to about 2 inches from the bottom of the closed end of the flexible bag 12, such as in the finger position 36.

In operation, when a patient inserts an appendage 11 into the flexible bag 12, excess material, such as finger portion 36 and thumb portion 32 may extend past the digits of the patient. To customize the length of the flexible bag 12, the excess material of the flexible bag 12 having the second fastening element 31 is folded towards the first fastening element 30 and the first fastening element 30 and the second fastening element 31 are engaged as shown in FIG. 7. This folded position causes the excess material of the flexible bag 12 to be restrained, thereby reducing the length and/or width of the extended position of the flexible bag.

First fastening element 30 and second fastening element 31 can be located at any location on the flexible bag 12 provided they are capable of being engaged. In another embodiment, multiple first fastening elements 30 engage multiple second fastening elements 31 to obtain a folded position of the flexible bag that has a length and/or width that is less than the length and/or width of he extended position of the flexible bag 12.

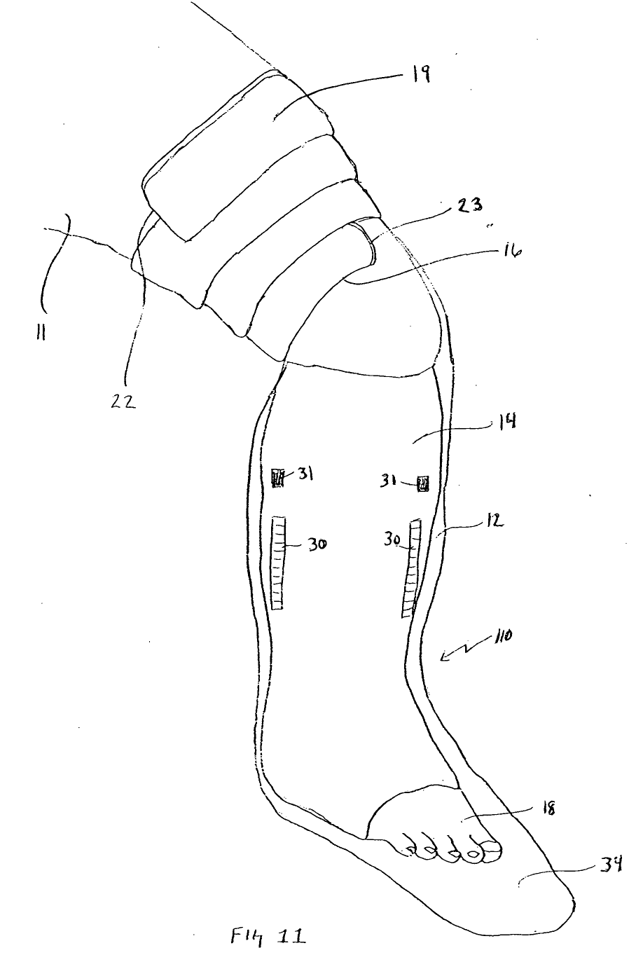

As shown in FIGS. 8A-12B, the process as described above can be used to cover a leg injury. As shown in FIG. 8A, the flexible bag 12 functions as described above except that a foot portion 34 is defined rather than the finger portion 36 or thumb portion 32. As shown in FIG. 8B, the flexible bag 12 may comprise a plurality of first fastening elements 30 and a plurality of second fastening elements 31. As shown in FIG. 10B, the flexible bag 12 may comprise a first fastening element 30 and a second fastening element 31 on a first side of the patient's appendage 11 and a first fastening element 30 and a second fastening element 31 on a second side of the patient's appendage 11.

As shown in FIGS. 10A-12B, in order to effectuate a water resistant seal, a first end 23 of the sealing band 16 is first wrapped around the patient's appendage or leg 11, in a first direction and fastened to the sealing band anchor as shown in FIGS. 10A and 10B. The second end 22 is subsequently wrapped around the patient's appendage 11 in a second direction that is generally opposite from the first direction. As the second end 22 is wrapped around the appendage 11, each successive wrap or coil overlaps at least a part of the previous coil of the sealing band 16, thereby creating a “shingling effect.” In one embodiment, the substantially flat side 41 is adjacent the skin of the patient and the opposing portion 19 is located on the exterior of the coiled sealing band 16. In one embodiment, when affixing a flexible bag 12 for a leg injury the sealing band 16 is wrapped around the thigh area of the leg rather than below the knee to provide a better sealing arrangement.

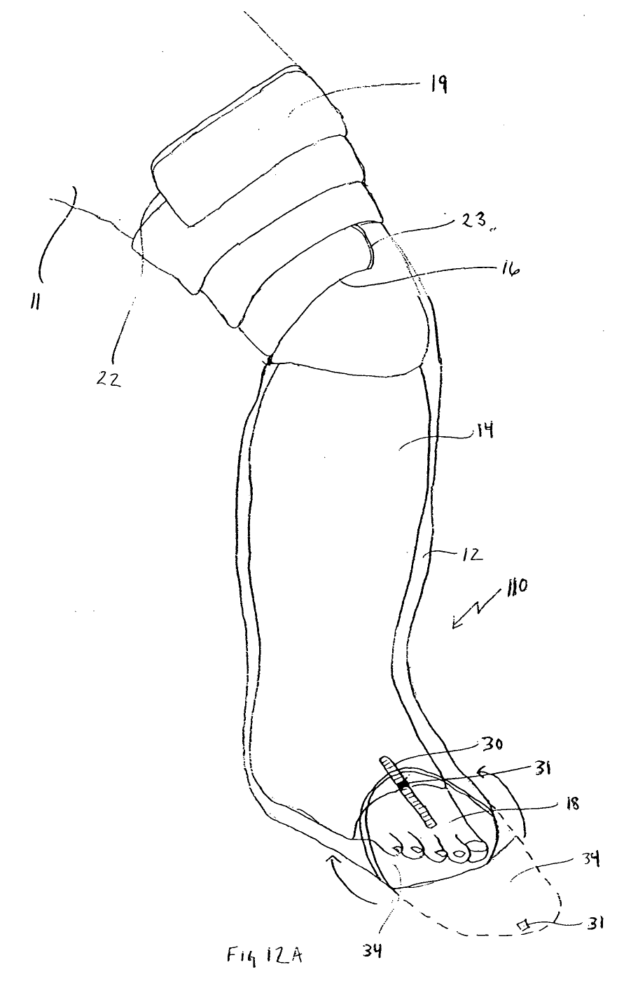

As shown in FIG. 11, once the remaining length of the sealing band 16 is wound around the patient's appendage 11, the hooked material 17B is attached to the loop material 19 of the top portion of the sealing band 16.

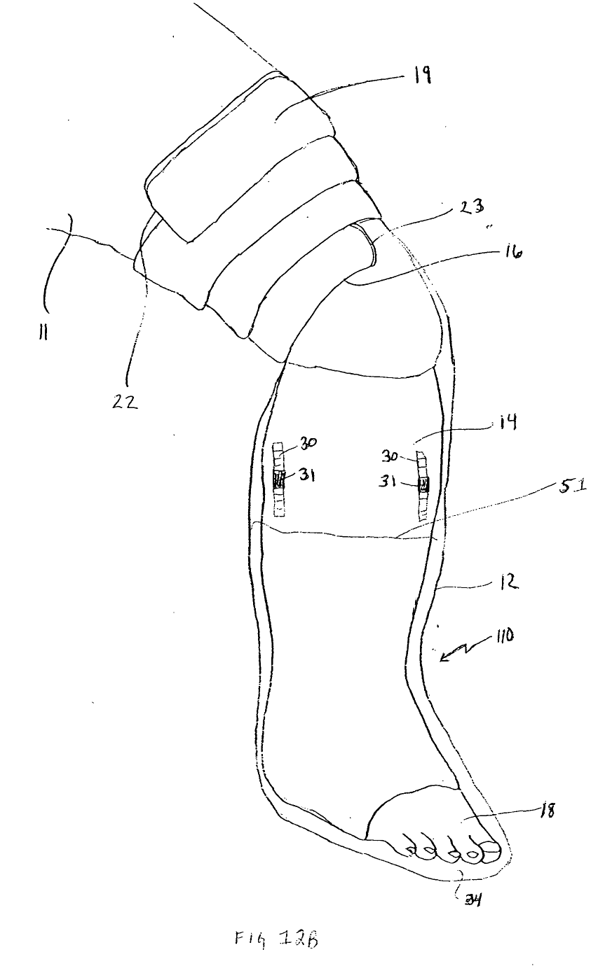

As shown in FIGS. 12A and 12B, to customize a dimension of the flexible bag 12, the excess material of the flexible bag 12 having the second fastening element 31 is folded towards the first fastening element 30 and the first fastening element 30 and the second fastening element 31 are engaged. As shown in FIG. 12A, the excess material is folded and fastened above or below the foot area 34. As shown in FIG. 12B, the excess material is folded at seam 51 and the first fastening element 30 and the second fastening element 31 on the first side of the patient's appendage are engaged and the first fastening element 30 and the second fastening element 31 on the second side of the patient's appendage are engaged.

Accordingly, the adjustable protective covers of the present invention are effective for a larger group of patients of different sizes. The present designs provide the ability of a patient, with normal functionality, to apply the protective cover 10, 110 without the need of aid from another person under normal conditions. The present invention also provides a “one size fits all” adult adjustable protective cover, and a “one size fits all” pediatric adjustable cover, each having water resistant integrity and the ability to cover plaster casts and synthetic casts, braces, PICC lines, Infusion Therapy Sites, active IV sites, bandages, surgical dressings, compressions wraps/bandages, prostheses, splints, lacerations, sutures, burns, rashes, allergic reactions, diabetic ulcers and skin grafts without having the protective material of the flexible bag resting snugly against the affected area or rubbing against the affected area either while applying the protective cover 10, 110 or once the protective cover 10, 110 has been applied.

Whereas particular embodiments of this invention have been described above for purposes of illustration, it will be evident to those skilled in the art that numerous variations of the details of the present invention may be made without departing from the invention.

What is claimed is:

1. An adjustable protective cover for a medical device or wound comprising:
an elongated water resistant flexible bag adjustable to an extended position and a folded position and having an opening on at least one end capable of housing at least part of a patient's appendage and a medical device or wound in the interior of the flexible bag.

2. The adjustable protective cover according to claim 1, wherein the flexible bag has a folded length and/or width that is smaller than an extended length and/or width of the flexible bag.

3. The adjustable protective cover according to claim 1, wherein the flexible bag in the extended position has a length from about 5 inches to about 48 inches.

4. The adjustable protective cover according to claim 1, wherein the flexible bag in the folded position has a folded length of from less than about 2 inches to about 20 inches smaller than an extended length of the flexible bag in the extended position.

5. The adjustable protective cover according to claim 1, wherein the flexible bag in the extended position has a width of from about 2 inches to about 18 inches.

6. The adjustable protective cover according to claim 1, wherein the flexible bag in the folded position has a folded width of from less than about 2 inches to about 12 inches smaller than an extended width of the flexible bag in the extended position.

7. The adjustable protective cover according to claim 1, wherein the opening of the flexible bag is at least partially surrounded by an elastic material.

8. The adjustable protective cover according to claim 7, wherein the opening has a width of from about 3 inches to about 5 inches when the elastic material is in a contracted position and from about 6 inches to about 14 inches when the elastic material is in an extended position.

9. The adjustable protective cover according to claim 1, further comprising:
at least one first bag fastening element disposed on a surface of the flexible bag; and
at least one second bag fastening element disposed on a surface of the flexible bag, the second bag fastening element capable of being engaged by the first bag fastening element.

10. The adjustable protective cover according to claim 9, wherein the first bag fastening element and the second bag fastening element are disengaged in the extended position and engaged in the folded position.

11. The adjustable protective cover according to claim 9, wherein the first fastening element and the second fastening element comprise hook and loop material, removable adhesive, snaps, hook and eye fasteners, buttons and/or magnets.

12. The adjustable protective cover according to claim 9, wherein the first bag fastener and the second bag fastener are both located on the same exterior surface of the flexible bag.

13. The adjustable protective cover according to claim 9, wherein the first bag fastener is located adjacent a closed end of the flexible bag.

14. The adjustable protective cover according to claim 9, wherein a plurality of the first bag fasteners engage a plurality of the second bag fasteners.

15. The adjustable protective cover according to claim 1, wherein the flexible bag is foldable to a dimension that closely mimics a dimension of the patient's appendage.

16. The adjustable protective cover according to claim 1, wherein the cover is structured and arranged for covering a patient's arm.

17. The adjustable protective cover according to claim 1, wherein the cover is structured and arranged for covering a patient's leg.

18. The adjustable protective cover according to claim 1, further comprising a sealing band anchor adjacent the opening of the flexible bag, and an elongated substantially flat water-resistant sealing band having sufficient length to be wrapped around the patient's appendage at least once, the sealing band comprising:
at least one substantially flat side having a sealing band anchor attachment, the sealing band anchor attachment capable of engaging the sealing band anchor, and
a sealing band connector disposed on the substantially flat side capable of attaching to an area on an opposing side of the sealing band or the flexible bag to substantially seal the open end of the flexible bag against the intrusion of moisture.

19. The adjustable protective cover according to claim 18, wherein the sealing band comprises a substantially flat side comprising neoprene, waterproof foamed materials, latex, silicone based material and/or PVC, and an opposing side comprising a looped material.

20. An adjustable cover system for a medical device or wound comprising:
an elongated water resistant flexible bag adjustable to an extended position and a folded position and having an opening on at least one end capable of housing at least part of a patient's appendage and a medical device or wound in the interior of the flexible bag;
at least one first bag fastening element disposed on a surface of the flexible bag;
at least one second bag fastening element disposed on a surface of the flexible bag, the second bag fastening element capable of being engaged by the first bag fastening element;
at least one sealing band anchor disposed on an exterior surface of the flexible bag; and
an elongated water-resistant flexible substantially flat sealing band having sufficient length to be wrapped around the patient's appendage at least once, the sealing band comprising:
at least one substantially flat side having at least one portion capable of engaging the sealing band anchor, and
a sealing band connector disposed on the substantially flat side capable of attaching to an area on an opposing side of the sealing band or the flexible bag to substantially seal the open end of the flexible bag against the intrusion of moisture.

21. The protective cover according to claim 20, wherein the flexible bag has a folded length and/or width that is smaller than an extended length and/or width of the flexible bag.

22. The adjustable protective cover according to claim 20, wherein the first bag fastening element and the second bag fastening element are disengaged in the extended position and engaged in the folded position.

23. The adjustable protective cover according to claim 20, wherein the first bag fastener and the second bag fastener are both located on an exterior surface of the flexible bag.

24. The adjustable protective cover according to claim 20, wherein the first fastening element and the second fastening element comprise hook and loop material, removable adhesive, snaps, hook and eye fasteners, buttons and/or magnets.

25. The adjustable protective cover according to claim 20, wherein a plurality of the first bag fasteners engage a plurality of the second bag fasteners.

26. The adjustable protective cover according to claim 20, wherein the flexible bag is foldable to a dimension that closely mimics a dimension of the patient's appendage.

27. The adjustable protective cover according to claim 20, wherein the cover is structured and arranged for covering a patient's arm.

28. The adjustable protective cover according to claim 20, wherein the cover is structured and arranged for covering a patient's leg.

29. The adjustable protective cover according to claim 20, wherein the sealing band comprises a substantially flat side comprising neoprene, waterproof foamed materials, latex, silicone based material and/or PVC, and an opposing side comprising a looped material.

30. The adjustable protective cover according to claim 20, wherein at least a portion of the sealing band and the sealing band anchor engage by means of hook and loop material, removable adhesive, snaps, hook and eye fasteners, buttons and/or magnets.

31. A method of using an adjustable cover for a medical device or wound comprising:
providing an elongated water resistant flexible bag adjustable to an extended position and a folded position and having an opening on at least one end;
inserting a patient's appendage through the opening into the flexible bag;
sealing the opening against the patient's appendage; and
folding a portion of the bag from the extended position to the folded position.
